(19) United States
(12) Patent Application Publication
(10) Pub. No.: US 2023/0367271 A1
(43) Pub. Date: Nov. 16, 2023

(54) SYSTEMS AND METHODS FOR TIME-SERIES DATA PROCESSING IN MACHINE LEARNING SYSTEMS

(71) Applicant: Salesforce, inc., San Francisco, CA (US)

(72) Inventors: Fabian Ricardo Latorre Gomez, San Francisco, CA (US); ChengHao Liu, Singapore (SG); Doyen Sahoo, Singapore (SG); Chu Hong Hoi, Singapore (SG)

(21) Appl. No.: 17/947,605

(22) Filed: Sep. 19, 2022

Related U.S. Application Data
(60) Provisional application No. 63/364,697, filed on May 13, 2022.

(51) Int. Cl. G05B 13/02

(52) U.S. Cl. CPC G05B 13/027; G16H 50/70

(57) ABSTRACT

Embodiments described herein provide using a measure of distance between time-series data sequences referred to as optimal transport warping (OTW). Measuring the OTW distance between unbalanced sequences (sequences with different sums of their values) may be accomplished by including an unbalanced mass cost. The OTW computation may be performed using cumulative sums over local windows. Further, embodiments herein describe methods for dealing with time-series data with negative values. Sequences may be split into positive and negative components before determining the OTW distance. A smoothing function may also be applied to the OTW measurement allowing for a gradient to be calculated. The OTW distance may be used in machine learning tasks such as clustering and classification. An OTW measurement may also be used as an input layer to a neural network.

### CROSS REFERENCE(S)

The instant application is a nonprovisional of and claims priority under 35 U.S.C. 119 to U.S. provisional application No. 63/364,697, filed May 13, 2022, which is hereby expressly incorporated by reference herein in its entirety.

### TECHNICAL FIELD

The embodiments relate generally to machine learning systems, and more specifically to systems and methods for time-series data processing.

### BACKGROUND

Machine learning systems have been widely used in analysis of time-series data. Time-series data may often be used to train a machine learning system for time-series predictions, such as weather prediction, heart disease prediction based on electrocardiogram (ECG) data, time-series classification and/or the like. Due to the time-varying nature of the time-series data, Euclidean distances are not suitable as a similarity measure between time-series, as they can arbitrarily change when one of the inputs is time-shifted, e.g., from a first time period to a second time period. Other methods such as dynamic time-warping are computationally expensive.

Therefore, there is a need for improved systems and methods for time-series data processing.

### BRIEF DESCRIPTION OF THE DRAWINGS

FIG. 1 illustrates an example pair of time-series data.

FIG. 2A is an example logic flow diagram of a method for processing time-series data according to embodiments herein.

FIG. 2B is an example logic flow diagram of a method for processing time-series data according to embodiments herein.

FIG. 3 illustrates exemplary performance in using methods described herein for classification.

FIG. 4 illustrates exemplary performance in using methods described herein for clustering.

FIG. 5 illustrates an exemplary algorithm for using optimal transport warping as a neural network layer.

FIG. 6 illustrates synthetic datasets for testing performance of methods described herein.

FIG. 7 illustrates exemplary performance of methods described herein applied to an input to a neural network with synthetic datasets.

FIG. 8 illustrates exemplary performance of methods described herein applied to an input to a neural network with real datasets.

FIG. 9 is a simplified diagram illustrating a computing device implementing the time-series processing according to embodiments herein.

FIG. 10 is a simplified block diagram of a networked system suitable for implementing the optimal transport warping framework described herein.

Embodiments of the disclosure and their advantages are best understood by referring to the detailed description that follows. It should be appreciated that like reference numerals are used to identify like elements illustrated in one or more of the figures, wherein showings therein are for purposes of illustrating embodiments of the disclosure and not for purposes of limiting the same.

### DETAILED DESCRIPTION

As used herein, the term “network” may comprise any hardware or software-based framework that includes any artificial intelligence network or system, neural network or system and/or any training or learning models implemented thereon or therewith.

As used herein, the term “module” may comprise hardware or software-based framework that performs one or more functions. In some embodiments, the module may be implemented on one or more neural networks.

Time series data are a continuous-time signal, or more often discrete-time samples of a signal reflecting values of a variable at different time points over a time period, e.g., the ECG data of a patient, the stock price, etc. Due to the time-varying nature, Euclidean distances are not suitable as a similarity measure between time-series, as they can arbitrarily change when one of the inputs is time-shifted, e.g., from a first time period to a second time period. Other methods such as dynamic time-warping (DTW), which dynamically warps one sequence to optimally match another sequence, are computationally expensive. Using DTW as an input layer to a neural network, for example, may create a computational bottleneck as it is higher complexity than a regular fully-connected neural network.

Embodiments described herein provide systems and methods for measuring a distance between time-series datasets, which may be applied in a number of use cases.

In some embodiments, dubbed optimal transport warping (OTW) may be applied to measure the distance between two time series datasets. For example, for “unbalanced” datasets, in which the sums of values in different datasets can be different, an adjustment may be applied such that an unbalanced mass cost is added to the distance measurement to account for the difference in the sum of the values. A predetermined parameter may be configured which adjusts how much the unbalancedness affects the distance measurement. Computational efficiency may be improved by limiting the unbalanced optimal transport function to within a defined window size.

In another embodiment, when time-series data have negative values, the OTW distance may be measured by splitting the sequences into versions which contain only the negative and only the positive values respectively, then performing the measurement between those sequences.

In another embodiment, a smoothing function may be applied to the OTW measurement, which smooths the absolute value function to not have an instantaneous step, allowing for a gradient to be calculated.

In this way, the OTW-based distance measurement between time-series datasets inherit the properties from OTW. For example, OTW enjoys linear time and space (memory) complexity, is differentiable, and can be parallelized. OTW has a moderate sensitivity to time and shape distortions, making it ideal for time-series data. In addition, OTW has an advantage over DTW in that it obeys, at least in some embodiments, the triangle inequality. For example, OTW distance between time-series datasets A and B, added to the OTW distance between time-series datasets B and C is greater than the OTW distance between A and C. Obeying the triangle inequality makes at least some embodiments of OTW a true metric. Therefore, the OTW-based distance measurement between time-series data can achieve superior performance and also maintains computational efficiency.

Calculating a distance between time-series data may be useful in a number of scenarios. First, OTW distance may be used in classifying time-series data. For example, ECG data may be compared to known heart pulse shapes, and thereby classified, aiding in the interpretation of the ECG data. Second, OTW distance may be used in unsupervised training by determining clusters of time-series datasets that are “close” to one another based on the OTW distance. Third, OTW distance may be used as a layer in a neural network. For example, the first hidden layer of a neural network may consist of OTW distances between the input and the rows of a matrix. As the complexity of this layer is linear, it does not create a bottleneck as a DTW layer would create.

Therefore, the accuracy and efficiency in time-series data measurement may help to improve training performance and systems of time-series processing systems, such as a neural network-based prediction system that predicts the likelihood of a diagnostic result (e.g., specific heart beat patterns, etc.), a network monitor that predicts network traffic and delay over a time period, an electronic trading system that makes trading decisions based on time-series data reflecting market dynamics and portfolio performance over time, and/or the like.

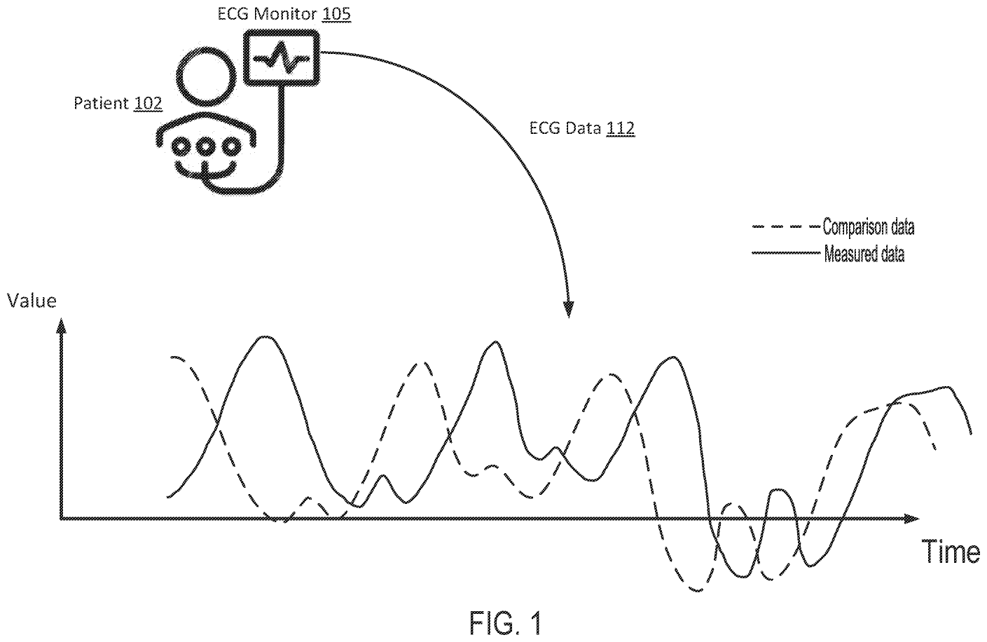

FIG. 1 illustrates an example user case of obtaining time-series data in a healthcare environment. For example, a patient 102 may be equipped with ECG sensors connected to an ECG monitor 105 which obtain the patient's ECG measurement data 112. For diagnostic purpose, time-series data for evaluation may comprise the patient's ECG measurement 112, and the other time-series data may represent a baseline sequence for comparison. The x-axis represents time in some units, and the y-axis represents the value of each dataset for each time index. The solid line represents one dataset and the dashed line represents another dataset. The two datasets illustrated are visually similar, although shifted in time. If one were to compute a distance measurement based on a simple value comparison at each time index, the result would be misleading, as the distance would be very large, even though the waveforms are similar, only shifted in time. For example, in ECG data, the shape of a waveform may be important, not the precise time in which that waveform appears in the data. The OTW distance measurement as described herein overcomes this limitation, and others when compared to other methods such as DTW.

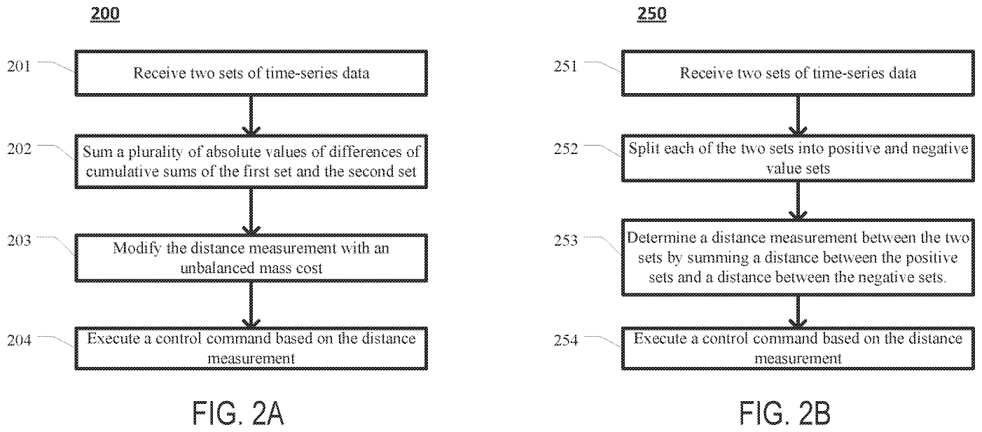

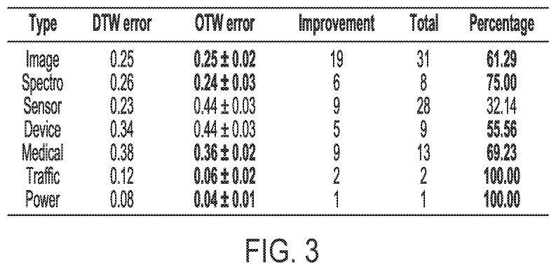

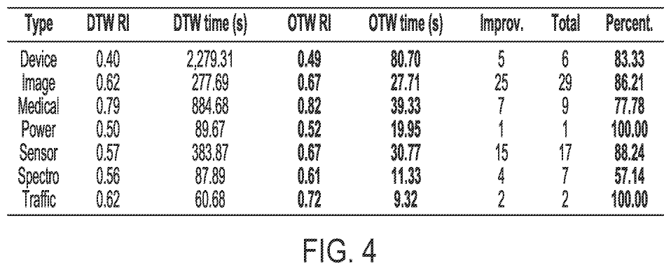

FIG. 2A is an example logic flow diagram illustrating a method 200 of optimal transport warping, according to some embodiments described herein. One or more of the processes of method 200 may be implemented, at least in part, in the form of executable code stored on non-transitory, tangible, machine-readable media that when run by one or more processors may cause the one or more processors to perform one or more of the processes. In some embodiments, method 200 corresponds to the operation of the OTW module 330 (e.g., FIGS. 3-4) that performs optimal transport warping.

As illustrated, the method 200 includes a number of enumerated steps, but aspects of the method 200 may include additional steps before, after, and in between the enumerated steps. In some aspects, one or more of the enumerated steps may be omitted or performed in a different order.

At step 201, the system receives two sets of time-series data, for example the datasets as illustrated in FIG. 1.

At step 202, the system sums a plurality of absolute values of differences of cumulative sums of the first set and the second set. In some embodiments, this may be represented as:

$${\sum }_{i = 1}^{n} \vert A ( i ) - B ( i ) \vert , where A ( i ) := {\sum }_{j = 1}^{i} {a}_{j} and B ( i ) := {\sum }_{j = 1}^{i} {b}_{j}$$

which is described in more detail below.

At step 203, the system modifies the distance measurement with an unbalanced mass cost. The unbalanced mass cost may be determined based on the difference between the sum of values of each set, multiplied by a predetermined constant. For example, if the sum of values in the first set equals the sum of values in the second set, the unbalanced mass cost would be zero, as the two sets would be balanced. In another example, if the sum of values in the first set is X more than the sum of values in the second set, then the unbalanced mass cost may be X multiplied by m, where m is a predetermined constant (hyper-parameter).

At step 204, the system executes a control command based on the distance measurement. For example, the system may display a classification such as a medical diagnostic recommendation based on one of the sequences on a user interface display when the time-series data represent patient measurement data.

In another example, the system process may be adjusted based on the control command, e.g., to generate and transmit an electronic trading order based on the distance measurement that indicates a portfolio return over a time period.

FIG. 2B is an example logic flow diagram illustrating a method 250 of optimal transport warping, according to some embodiments described herein. One or more of the processes of method 250 may be implemented, at least in part, in the form of executable code stored on non-transitory, tangible, machine-readable media that when run by one or more processors may cause the one or more processors to perform one or more of the processes. In some embodiments, method 250 corresponds to the operation of the OTW module 330 (e.g., FIGS. 3-4) that performs optimal transport warping.

As illustrated, the method 250 includes a number of enumerated steps, but aspects of the method 250 may include additional steps before, after, and in between the enumerated steps. In some aspects, one or more of the enumerated steps may be omitted or performed in a different order.

At step 251, the system receives two sets of time-series data, for example the datasets as illustrated in FIG. 1.

At step 252, the system splits each of the two sets into positive and negative value sets. For example, the first dataset may include positive and negative values, as illustrated in FIG. 1 where values go below the axis. The positive set may include the same amount of values, but with every negative value set to zero. Likewise, the negative set may include the same amount of values, but with every positive value set to zero. In this way, the length of the data set and the relative position of each of the values remains the same. In some embodiments, separate positive and negative value sets are generated only after a determination that the respective data sets include both positive and negative values. The individual positive and negative sets may be stored separately in memory, or the system may utilize the single data set with both positive and negative values as a single copy in memory, and when processing the positive or negative set, it may set the corresponding values to zero as a processing step.

At step 253, the system determines a distance measurement between the two sets by summing a distance between the positive sets and a distance between the negative sets.

At step 254, the system executes a control command based on the distance measurement. For example, the system may display a classification of one of the sequences on a user interface display. In another example, another system process may be adjusted based on the control command.

The methods described in FIGS. 2A and 2B are exemplary. Features from each of methods 200 and 250 may be combined in different ways, for example splitting sequences into positive and negative sequences may be performed together with adding an unbalanced mass cost. Additional adjustments may be made to the distance measurement in either of methods 200 or 250 which may improve performance further, as is described in more detail below. Further, not every embodiment may require each of the steps of methods 200 and 250. For example, in some embodiments the sets may not be split into positive and negative sets, even when one or both of the sets contain negative numbers. The following discussion presents a mathematical description of the features described above (splitting sets into negative and positive and unbalanced mass cost) in addition to further features such as smoothing.

First, consider a pair of time-series data sequences (sets) a and b which have only positive numbers, with n values each. A baseline (optimal transport) distance measurement between a and b may be defined as:

$$OTW ( a , b ) = {\sum }_{i = 1}^{n} \vert A ( i ) - B ( i ) \vert , where$$

$$A ( i ) := {\sum }_{j = 1}^{i} {a}_{j} and B ( i ) := {\sum }_{j = 1}^{i} {b}_{j}$$

where A and B in the equation above are the cumulative distribution functions of a and b respectively.

To account for unbalanced sets (sets with cumulative values that are not equal), additional changes may be made to the distance measurement formulation. An unbalanced mass cost may be introduced which adds to the distance defined above:

$$0 T {W}_{m} ( a , b ) = m \vert A ( n ) - B ( n ) \vert + {\sum }_{i = 1}^{n - 1} \vert A ( i ) - B ( i ) \vert$$

The parameter m in the equation above is a predetermined value (hyper-parameter). The parameter m may be adjusted based on how much it is desired that unbalancedness be penalized. For example, it may be determined based on cross-validation using a validation training set. In some embodiments, m is less than n (the number of values in each set) so as to not put too much weight on the unbalanced mass component. This unbalanced distance measurement increases linearly when a time-shift is introduced, making it ideal for time-series applications like demand forecasting, where a shift in time can represent a change in the seasonality of a product.

Another improvement may be to constrain the function to be local. Constraining the cumulation to be within a window may decrease the amount of necessary computation. Another parameter, s, may be introduced which may be used to adjust the level of localness which may be beneficial to change depending on the circumstance:

$${OTW}_{m , s} ( a , b ) = m \vert {A}_{s} ( n ) - {B}_{s} ( n ) \vert + {\sum }_{i = 1}^{n - 1} \vert {A}_{s} ( i ) - {B}_{s} ( i ) \vert$$

$${A}_{s} ( i ) := {\sum }_{j = 1}^{i} {a}_{j} - {\sum }_{j = 1}^{i - s} {a}_{j} and {B}_{s} ( i ) := {\sum }_{j = 1}^{i} {b}_{j} - {\sum }_{j = 1}^{i - s} {b}_{j}$$

The s parameters may be predetermined based on the desired level of localness. For example, it may be selected based on cross-validation using a validation training set.

The distance measurement may also be made differentiable by using a smoothed approximation of the absolute value functions, controlled by a parameter β:

$${OTW}_{m , s}^{β} ( a , b ) = {mL}_{β} ( {A}_{s} ( n ) - {B}_{s} ( n ) ) + {\sum }_{i = 1}^{n - 1} {L}_{β} ( {A}_{s} ( i ) - {B}_{s} ( i ) )$$

$${L}_{β} ( x ) = \{ \begin{matrix}{x}^{2} / ( 2 β ) & \vert x \vert < β \\ \vert x \vert - β / 2 & \vert x \vert \geq β\end{matrix}$$

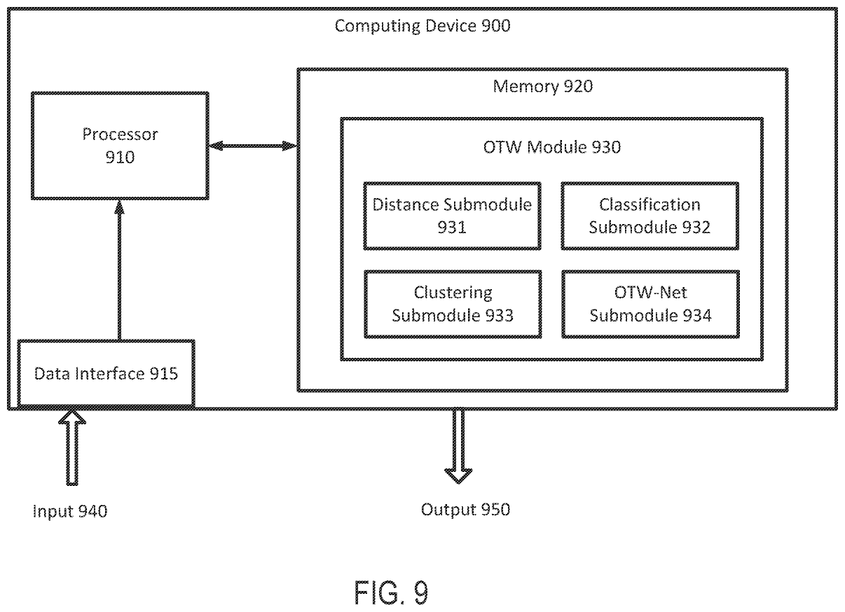

As β approaches zero, the smoothed absolute value function approaches the regulate absolute value function. With the function smoothed as in the equation above, a gradient may be computed, facilitating the use of the distance measurement in a neural network layer as discussed below in FIG. 9.

The distance measurement may be extended to sets with negative values. In some embodiments, the equations above may be applied without change to time index data sets with negative values without any changes. In other embodiments, the distance measurement function may be modified as discussed with respect to steps 202 and 203. Specifically, sequences a and b may be split into their positive and negative parts. This may be represented as a₊=max(a, 0) and a₋=max(−a, 0) for each element. After splitting the sequences, the unbalanced OTW distance between the positive and negative parts may be summed together:

OTW_{m,s}^{β}(a, b)=OTW_{m,s}^{β}(a₊, b₊)+OTW_{m,s}^{β}(a₋, b₋)

As described above, there are multiple features which may be used as part of the distance measurement. Specifically, unbalanced mass cost, localness, differentiable, and using negative values. These features may be used in different combinations, as they do not all rely on each other. For example, an OTW distance measurement may be performed with an unbalanced mass cost, using a smoothed absolute value approximation to make it differentiable, while only using positive value sequences and not constraining the sums to be local. OTW distance measurements provide a flexible way to measure distance, with the beneficial properties detailed above, while maintaining lower computational complexity than alternative methods like dynamic transport warping (DTW).

One practical application of measuring distance using OTW is in grouping sequences into classes. If a sequence is determined to be a member of a class based on an OTW distance measurement, a system may perform an action based on that determination. For example, a control command (such as providing an indication or performing an action in a mechanical system) may be executed by a system based on the classification of a time-series data set.

FIG. 3 illustrates exemplary performance in using methods described herein for classification. Specifically, illustrated is a comparison between DTW and OTW for a 1-nearest-neighbor classification task. In 1-nearest-neihbgor, a sequence is classified based on the nearest sequence in a training set. Classifiers were trained on the UCR time series classification archive, which consists of a large number of univariate time series data. Due to lower complexity of OTW compared to DTW, it runs considerably faster, and may therefore be considered superior to DTW even with the same classification performance. As shown, there is a noticeable improvement in each category except the sensor category.

Another practical application of OTW distance measurement is in hierarchical clustering. For example, in unsupervised learning, time-series data sets may be grouped based on OTW distance, providing information about the group relationships of groups of sequences.

FIG. 4 illustrates exemplary performance in using methods described herein for clustering. Again, DTW and OTW distance are compared. A clustering algorithm was run on a time-series benchmark collection. The quality of clustering is evaluated using the Rand Index (RI), which is a measure of the similarity between two data clusterings. As shown in the table for FIG. 4, OTW distance outperforms DTW on most datasets considered. This was achieved with less time required for the method to run, based on the lower computational requirements. On some datasets like Image, Traffic, and Sensor, the advantage is apparent, illustrating that based on the type of sequences considered, OTW may be especially well-suited.

OTW distance may also be employed to design neural network layers that are better suited for time series data. For example, a neural network may be designed in which the first hidden layer consists of OTW distances between the input and the rows of a matrix, which is the trainable parameter of the layer. When there are k such rows, then the computational complexity is O(kn), where n is the length of the input. On top of such features an arbitrary network architecture may be added, which outputs the class probabilities. A typical multi-layer fully-connected neural network also has a complexity for each linear layer of O(kn). By having the same complexity, using OTW distance is possible without creating a bottleneck for computation. This is in contrast to a similarly designed network with DTW used for the first hidden layer. A DTW-based layer has a complexity of O(kn²) which creates a bottleneck for data.

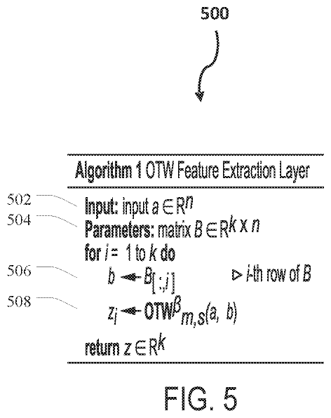

FIG. 5 illustrates an exemplary algorithm 500 for using optimal transport warping as a neural network layer. One or more of the processes of algorithm 500 may be implemented, at least in part, in the form of executable code stored on non-transitory, tangible, machine-readable media that when run by one or more processors may cause the one or more processors to perform one or more of the processes. In some embodiments, algorithm 500 corresponds to an example operation of the OTW-Net submodule 934 of FIG. 9.

As shown at line 502, a time-series data set “a” of length n is the input. As shown at line 504, the parameters are in the form of matrix B which is a k by n matrix. As shown at line 506, each row of matrix B defines a sequence b. As shown at line 508, the output z for each b is the OTW distance between a and b. The version of OTW which is shown is specifically OTW_{m,s}^{β} which includes smoothing, making the distance measurement differentiable. As such, a gradient may be calculated allowing for gradient descent to be used to update the parameters of matrix B via back-propagation.

The output z may be used as the input to a neural network, which may produce some output. For example, the neural network may provide some output that classifies input a. The output of the neural network may be used to execute some control command in a dynamic system.

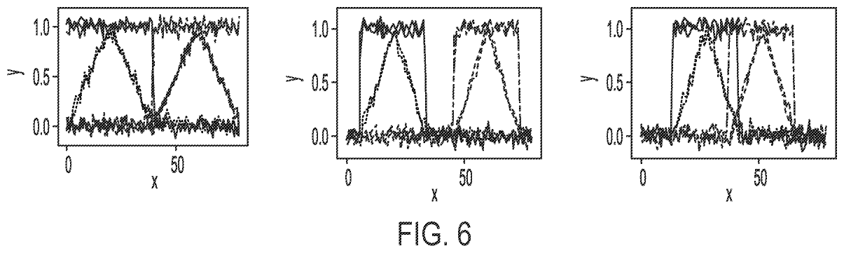

FIG. 6 illustrates synthetic datasets for testing performance of methods described herein. The three synthetic datasets illustrated each consist of four different classes of sequences determined by a combination of shape (square/triangle) and time shift. Each dash type corresponds to a different class. Each of the three plots shows sequences of the same four classes, each with slight modifications but with the major features making them part of the class being the same.

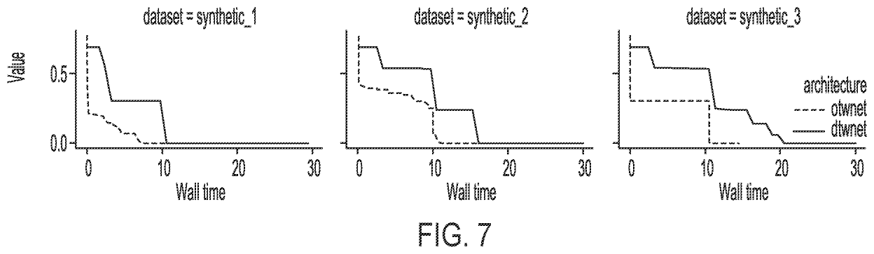

FIG. 7 illustrates exemplary performance of methods described herein applied to an input to a neural network with synthetic datasets such as those illustrated in FIG. 6. Specifically, test error is plotted against wall clock time (in seconds) for neural network classifiers. Again, a DTW based implementation is compared to an OTW based implementation. For the synthetic data experiment, the hidden layer sizes for both the DTW network and the OTW network were set to be the same. Both networks were trained for 500 epochs, Due to the computational bottleneck in the DTW network, its training time is orders of magnitude larger than the OTW network. Even though the DTW network converges in fewer epochs, it is not enough to offset the slower time-per-epoch it takes. In each of the tested cases, the OTW network achieved zero error in 50 to 60 percent of the time of the DTW network. This shows that the linear complexity of OTW is able to achieve the same or better performance as a DTW implementation while using less computer resources.

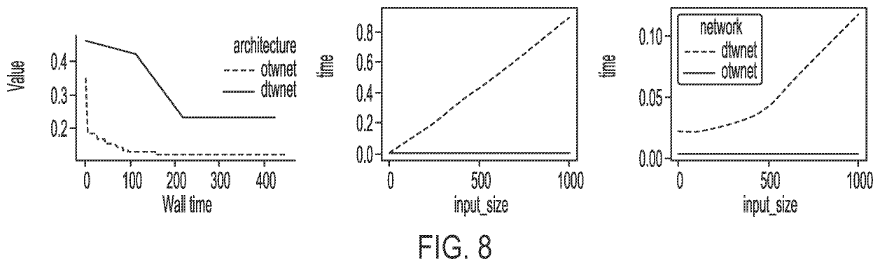

FIG. 8 illustrates exemplary performance of methods described herein applied to an input to a neural network with real datasets instead of synthetic datasets. Specifically, an OTW based neural network is again compared to a DTW based neural network. For this experiment, the hidden layer sizes for the OTW network were set as [500,500,500] and for the DTW network as [100,500,500]. The smaller size of the first hidden layer of the DTW network allowed for training in a reasonable amount of time. Both networks were trained for 5000 epochs. The first plot on the left illustrates the test error vs the training time. This illustrates that the quadratic complexity of the first layer in a DTW network makes this approach unfeasible on realistic datasets, and that one way to solve this problem is to use an OTW network architecture. The center plot illustrates wall clock time of a forward/backward pass of the neural network in a CPU as a function of the size of the input. The right plot illustrates wall clock time of a forward/backward pass of the neural network in a GPU as a function of the size of the input. As illustrated, there is a stark difference in time, and the OTW network runs considerably faster than the DTW based networks, both in CPU and GPU.

FIG. 9 is a simplified diagram illustrating a computing device implementing the OTW architecture described in FIGS. 1-8, according to one embodiment described herein. As shown in FIG. 9, computing device 900 includes a processor 910 coupled to memory 920. Operation of computing device 900 is controlled by processor 910. And although computing device 900 is shown with only one processor 910, it is understood that processor 910 may be representative of one or more central processing units, multi-core processors, microprocessors, microcontrollers, digital signal processors, field programmable gate arrays (FPGAs), application specific integrated circuits (ASICs), graphics processing units (GPUs) and/or the like in computing device 900. Computing device 900 may be implemented as a stand-alone subsystem, as a board added to a computing device, and/or as a virtual machine.

Memory 920 may be used to store software executed by computing device 900 and/or one or more data structures used during operation of computing device 900. Memory 920 may include one or more types of machine-readable media. Some common forms of machine-readable media may include floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

Processor 910 and/or memory 920 may be arranged in any suitable physical arrangement. In some embodiments, processor 910 and/or memory 920 may be implemented on a same board, in a same package (e.g., system-in-package), on a same chip (e.g., system-on-chip), and/or the like. In some embodiments, processor 910 and/or memory 920 may include distributed, virtualized, and/or containerized computing resources. Consistent with such embodiments, processor 910 and/or memory 920 may be located in one or more data centers and/or cloud computing facilities.

In some examples, memory 920 may include non-transitory, tangible, machine readable media that includes executable code that when run by one or more processors (e.g., processor 910) may cause the one or more processors to perform the methods described in further detail herein. For example, as shown, memory 920 includes instructions for OTW module 930 that may be used to implement and/or emulate the systems and models, and/or to implement any of the methods described further herein. An OTW module 930 may receive input 940 such as an input time-series data sequences (e.g., sequences as shown in FIG. 1), which may include training data with labelled classes (e.g., sequences as shown in FIG. 6), via the data interface 915. Input 940 may also be a system status variable which is sampled by computing device 900 in order to generate a time-series sequence. OTW module 930 may generate an output 950 which may be a distance measurement between time-series data sequences, classification of a time-series data sequence, a control signal, etc.

The data interface 915 may comprise a communication interface, a user interface (such as a voice input interface, a graphical user interface, and/or the like). For example, the computing device 900 may receive the input 940 (such as a training dataset) from a networked database via a communication interface. Or the computing device 900 may receive the input 940, such as time-series data sequences, from a user via the user interface.

In some embodiments, the OTW module 930 is configured to compute distances between time-series data sequences, and in some embodiments perform some action based on the measured distance. The OTW module 930 may further include a distance submodule 931 which performs the OTW distance measurement as described herein (e.g., with reference to FIGS. 1-2). The OTW module 930 may further includes submodules for implementing OTW measurements into different applications. Specifically, a classification submodule 932 may classify time-series data sequences as described with reference to FIG. 3. A clustering submodule 933 may group time-series data sequences into clusters as described with reference to FIG. 4. An OTW-Net submodule may include an OTW-based layer of in a neural network as described with reference to FIGS. 5-8. In one embodiment, the OTW module 930 and its submodules 931-934 may be implemented by hardware, software and/or a combination thereof.

Some examples of computing devices, such as computing device 900 may include non-transitory, tangible, machine readable media that include executable code that when run by one or more processors (e.g., processor 910) may cause the one or more processors to perform the processes of method. Some common forms of machine-readable media that may include the processes of method are, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

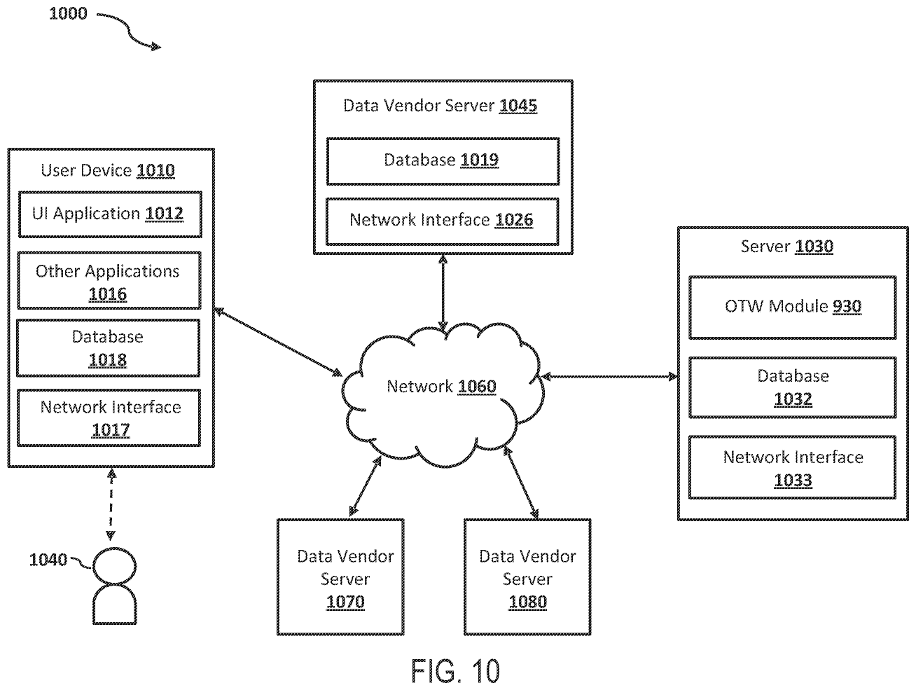

FIG. 10 is a simplified block diagram of a networked system 1000 suitable for implementing the OTW framework described in FIGS. 1-9 and other embodiments described herein. In one embodiment, system 1000 shows a system including the user device 1010 which may be operated by user 1040, data vendor servers 1045, 1070 and 1080, server 1030, and other forms of devices, servers, and/or software components that operate to perform various methodologies in accordance with the described embodiments. Exemplary devices and servers may include device, stand-alone, and enterprise-class servers which may be similar to the computing device 900 described in FIG. 9, operating an OS such as a MICROSOFT® OS, a UNIX® OS, a LINUX® OS, or other suitable device and/or server-based OS. It can be appreciated that the devices and/or servers illustrated in FIG. 10 may be deployed in other ways and that the operations performed, and/or the services provided by such devices and/or servers may be combined or separated for a given embodiment and may be performed by a greater number or fewer number of devices and/or servers. One or more devices and/or servers may be operated and/or maintained by the same or different entities.

The user device 1010, data vendor servers 1045, 1070 and 1080, and the server 1030 may communicate with each other over a network 1060. User device 1010 may be utilized by a user 1040 (e.g., a driver, a system admin, etc.) to access the various features available for user device 1010, which may include processes and/or applications associated with the server 1030 to receive an output data anomaly report.

User device 1010, data vendor server 1045, and the server 1030 may each include one or more processors, memories, and other appropriate components for executing instructions such as program code and/or data stored on one or more computer readable mediums to implement the various applications, data, and steps described herein. For example, such instructions may be stored in one or more computer readable media such as memories or data storage devices internal and/or external to various components of system 1000, and/or accessible over network 1060.

User device 1010 may be implemented as a communication device that may utilize appropriate hardware and software configured for wired and/or wireless communication with data vendor server 1045 and/or the server 1030. For example, in one embodiment, user device 1010 may be implemented as an autonomous driving vehicle, a personal computer (PC), a smart phone, laptop/tablet computer, wristwatch with appropriate computer hardware resources, eyeglasses with appropriate computer hardware (e.g., GOOGLE GLASS®), other type of wearable computing device, implantable communication devices, and/or other types of computing devices capable of transmitting and/or receiving data, such as an IPAD® from APPLE®. Although only one communication device is shown, a plurality of communication devices may function similarly.

User device 1010 of FIG. 10 contains a user interface (UI) application 1012, and/or other applications 1016, which may correspond to executable processes, procedures, and/or applications with associated hardware. For example, the user device 1010 may receive a message indicating the classification of a time-series data sequence from the server 1030 and display the message via the UI application 1012. In other embodiments, user device 1010 may include additional or different modules having specialized hardware and/or software as required.

In various embodiments, user device 1010 includes other applications 1016 as may be desired in particular embodiments to provide features to user device 1010. For example, other applications 1016 may include security applications for implementing client-side security features, programmatic client applications for interfacing with appropriate application programming interfaces (APIs) over network 1060, or other types of applications. Other applications 1016 may also include communication applications, such as email, texting, voice, social networking, and IM applications that allow a user to send and receive emails, calls, texts, and other notifications through network 1060. For example, the other application 1016 may be an email or instant messaging application that receives a prediction result message from the server 1030. Other applications 1016 may include device interfaces and other display modules that may receive input and/or output information. For example, other applications 1016 may contain software programs for asset management, executable by a processor, including a graphical user interface (GUI) configured to provide an interface to the user 1040 to view information based on an OTW measurement.

User device 1010 may further include database 1018 stored in a transitory and/or non-transitory memory of user device 1010, which may store various applications and data and be utilized during execution of various modules of user device 1010. Database 1018 may store user profile relating to the user 1040, predictions previously viewed or saved by the user 1040, historical data received from the server 1030, and/or the like. In some embodiments, database 1018 may be local to user device 1010. However, in other embodiments, database 1018 may be external to user device 1010 and accessible by user device 1010, including cloud storage systems and/or databases that are accessible over network 1060.

User device 1010 includes at least one network interface component 1017 adapted to communicate with data vendor server 1045 and/or the server 1030. In various embodiments, network interface component 1017 may include a DSL (e.g., Digital Subscriber Line) modem, a PSTN (Public Switched Telephone Network) modem, an Ethernet device, a broadband device, a satellite device and/or various other types of wired and/or wireless network communication devices including microwave, radio frequency, infrared, Bluetooth, and near field communication devices.

Data vendor server 1045 may correspond to a server that hosts database 1019 to provide training datasets including time-series data to the server 1030. The database 1019 may be implemented by one or more relational database, distributed databases, cloud databases, and/or the like.

The data vendor server 1045 includes at least one network interface component 1026 adapted to communicate with user device 1010 and/or the server 1030. In various embodiments, network interface component 1026 may include a DSL (e.g., Digital Subscriber Line) modem, a PSTN (Public Switched Telephone Network) modem, an Ethernet device, a broadband device, a satellite device and/or various other types of wired and/or wireless network communication devices including microwave, radio frequency, infrared, Bluetooth, and near field communication devices. For example, in one implementation, the data vendor server 1045 may send asset information from the database 1019, via the network interface 1026, to the server 1030.

The server 1030 may be housed with the OTW module 930 and its submodules described in FIG. 1. In some implementations, module 930 may receive data from database 1019 at the data vendor server 1045 via the network 1060 to generate outputs. The generated outputs may also be sent to the user device 1010 for review by the user 1040 via the network 1060.

The database 1032 may be stored in a transitory and/or non-transitory memory of the server 1030. In one implementation, the database 1032 may store data obtained from the data vendor server 1045. In one implementation, the database 1032 may store parameters of the OTW module 930. In one implementation, the database 1032 may store previously generated measurements, and the corresponding input feature vectors.

In some embodiments, database 1032 may be local to the server 1030. However, in other embodiments, database 1032 may be external to the server 1030 and accessible by the server 1030, including cloud storage systems and/or databases that are accessible over network 1060.

The server 1030 includes at least one network interface component 1033 adapted to communicate with user device 1010 and/or data vendor servers 1045, 1070 or 1080 over network 1060. In various embodiments, network interface component 1033 may comprise a DSL (e.g., Digital Subscriber Line) modem, a PSTN (Public Switched Telephone Network) modem, an Ethernet device, a broadband device, a satellite device and/or various other types of wired and/or wireless network communication devices including microwave, radio frequency (RF), and infrared (IR) communication devices.

Network 1060 may be implemented as a single network or a combination of multiple networks. For example, in various embodiments, network 1060 may include the Internet or one or more intranets, landline networks, wireless networks, and/or other appropriate types of networks. Thus, network 1060 may correspond to small scale communication networks, such as a private or local area network, or a larger scale network, such as a wide area network or the Internet, accessible by the various components of system 1000.

This description and the accompanying drawings that illustrate inventive aspects, embodiments, implementations, or applications should not be taken as limiting. Various mechanical, compositional, structural, electrical, and operational changes may be made without departing from the spirit and scope of this description and the claims. In some instances, well-known circuits, structures, or techniques have not been shown or described in detail in order not to obscure the embodiments of this disclosure. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims, and it is appropriate that the claims be construed broadly and, in a manner, consistent with the scope of the embodiments disclosed herein.

What is claimed is:

1. A method for measuring time-series data, the method comprising:
receiving a first set of time-series data corresponding to a first system status variable over a first period of time;
receiving a second set of time-series data corresponding to a second system status variable over a second period of time;
determining a distance measurement between the first set and a second set, the determining comprising:
summing a plurality of absolute values of differences of cumulative sums of the first set and the second set to provide the distance measurement; and
modifying the distance measurement with an unbalanced mass cost computed based on a difference between a first sum of values in the first set and a second sum of values in the second set; and
executing a control command pertaining to the first system status variable or the second system status variable based on the distance measurement.

2. The method of claim 1, wherein:
the cumulative sums of the first set and the second set are partial cumulative sums with a predetermined window size,
the first sum of values in the first set is a sum of a first subset of the values in the first set, and
the second sum of values in the second set is a sum of a second subset of the values in the second set.

3. The method of claim 1, wherein the plurality of absolute values are smoothed approximations of absolute values.

4. The method of claim 3, wherein:
the second set of time series data is comprised of trainable parameters,
the distance measurement is input to a neural network, and
the executing the control command is further based on an output of the neural network.

5. The method of claim 1, wherein at least one of the first set or the second set contains positive and negative values.

6. The method of claim 5, further comprising:
in response to determining that the first set or the second set contains both positive and negative values, splitting the first set into a first subset of all positive values and a second subset of all negative values and the second set into a third subset of all positive values and a fourth subset of all negative values;
wherein determining the distance measurement comprises determining a first distance measurement between the first and third subsets and determining a second distance measurement between the second and third subsets.

7. The method of claim 1, further comprising:
associating the first set with a class of time-series data based on the distance measurement.

8. The method of claim 1, further comprising:
associating the first set and the second set together in a cluster of time-series data sets based on the distance measurement.

9. A system for measuring time-series data, the system comprising:
a memory that stores a plurality of processor executable instructions;
a communication interface that receives a first set of time-series data corresponding to a first system status variable over a first period of time; and
one or more hardware processors that read and execute the plurality of processor-executable instructions from the memory to perform operations comprising:
receiving a second set of time-series data corresponding to a second system status variable over a second period of time;
in response to determining that the first set or the second set contains both positive and negative values, splitting the first set into a first subset of all positive values and a second subset of all negative values and the second set into a third subset of all positive values and a fourth subset of all negative values;
summing a plurality of absolute values of differences of cumulative sums of the first subset and the third subset to provide a first distance measurement;
summing a plurality of absolute values of differences of cumulative sums of the second subset and the fourth subset to provide a second distance measurement;
adding the first distance measurement and the second distance measurement to provide a composite distance measurement; and
executing a control command pertaining to the first system status variable or the second system status variable based on the composite distance measurement.

10. The system of claim 9, wherein the operations further comprise:
modifying the composite distance measurement with an unbalanced mass cost computed based on a difference between a first sum of values in the first set and a second sum of values in the second set.

11. The system of claim 9, wherein the cumulative sums of the first, second, and third subsets are partial cumulative sums with a predetermined window size.

12. The system of claim 9, wherein:
the plurality of absolute values of differences of cumulative sums of the first subset and the third subset are smoothed approximations of absolute values, and
the plurality of absolute values of differences of cumulative sums of the second subset and the fourth subset are smoothed approximations of absolute values.

13. The system of claim 12, wherein:
the second set of time series data is comprised of trainable parameters,
the composite distance measurement is input to a neural network, and
the executing the control command is further based on an output of the neural network.

14. The system of claim 9, wherein the operations further comprise:
associating the first set with a class of time-series data based on the composite distance measurement.

15. The system of claim 9, wherein the operations further comprise:
associating the first set and the second set together in a cluster of time-series data sets based on the composite distance measurement.

16. A non-transitory machine-readable medium comprising a plurality of machine-executable instructions which, when executed by one or more processors, are adapted to cause the one or more processors to perform operations comprising:
receiving a first set of time-series data corresponding to a first system status variable over a first period of time;
receiving a second set of time-series data corresponding to a second system status variable over a second period of time;
determining a distance measurement between the first set and a second set, the determining comprising:
summing a plurality of absolute values of differences of cumulative sums of the first set and the second set to provide the distance measurement; and
modifying the distance measurement with an unbalanced mass cost computed based on a difference between a first sum of values in the first set and a second sum of values in the second set; and
executing a control command pertaining to the first system status variable or the second system status variable based on the distance measurement.

17. The non-transitory machine-readable medium of claim 16, wherein:
the cumulative sums of the first set and the second set are partial cumulative sums with a predetermined window size,
the first sum of values in the first set is a sum of a first subset of the values in the first set, and
the second sum of values in the second set is a sum of a second subset of the values in the second set.

18. The non-transitory machine-readable medium of claim 16, wherein the plurality of absolute values are smoothed approximations of absolute values.

19. The non-transitory machine-readable medium of claim 18, wherein:
the second set of time series data is comprised of trainable parameters,
the distance measurement is input to a neural network, and
the executing the control command is further based on an output of the neural network.

20. The non-transitory machine-readable medium of claim 16, wherein at least one of the first set or the second set contains positive and negative values.
